(19) United States
(12) Patent Application Publication
(10) Pub. No.: US 2021/0186753 A1
(43) Pub. Date: Jun. 24, 2021

(54) LASER TREATMENT OF MEDIA OPACITIES

(71) Applicant: Alcon Inc., Fribourg (CH)

(72) Inventors: Muhammad K. Al-Qaisi, Ladera Ranch, CA (US); Paul Hallen, Colleyville, TX (US)

(21) Appl. No.: 17/126,899

(22) Filed: Dec. 18, 2020

Related U.S. Application Data
(60) Provisional application No. 62/950,560, filed on Dec. 19, 2019.

(51) Int. Cl. A61F 9/008

(52) U.S. Cl. CPC A61F 9/008; A61F 2009/00878; A61F 2009/00851; A61F 2009/00846

(57) ABSTRACT

The present disclosure provides a laser treatment system that includes an optical coherence tomography (OCT) imaging system that generates a plurality of profile depth scans and executes instructions on a processor to detect a position, a volume, or a combination thereof, of a media opacity in an eye based on the plurality of profile depth scans. The system further includes a three-dimensional (3D) eye tracker that executes instructions on the processor to track the position, the volume, or a combination thereof, of the media opacity in the eye based on the plurality of profile depth scans. The system also includes a laser system that includes a treatment laser and that precisely targets a plurality of ultra-short laser pulses generated by the treatment laser at the media opacity in the eye to at least partially remove the media opacity.

### TECHNICAL FIELD

The present disclosure relates to vitreoretinal surgery and surgical equipment, and more specifically, to a laser treatment system to improve removal of media opacities for vitreoretinal surgery and associated methods.

### BACKGROUND

Ophthalmic surgery is surgery performed on the eye or any part of the eye. Ophthalmic surgery saves and improves the vision of tens of thousands of patients every year. However, given the sensitivity of vision to even small changes in the eye and the minute and delicate nature of many eye structures, ophthalmic surgery is difficult to perform and the reduction of even minor or uncommon surgical errors or modest improvements in accuracy of surgical techniques can make an enormous difference in the patient's vision after the surgery.

One type of ophthalmic surgery, vitreoretinal surgery, encompasses various delicate procedures involving internal portions of the eye, such as the vitreous humor, the retina, and the vitreoretinal membrane. During ophthalmic surgery, such as vitreoretinal surgery, an ophthalmologist typically uses a non-electronic, optical, surgical microscope with oculars to view a magnified image of the eye undergoing surgery. More recently, vitreoretinal surgeons may use an ocular-free digital visualization system to aid visualization during vitreoretinal surgery such as an NGENUITY® (Novartis AG Corp., Switzerland) 3D Visualization System. These systems may include a three-dimensional (3D) high dynamic range (“HDR”) camera system with a pair of two-dimensional (2D) complementary metal-oxide-semiconductor (CMOS) sensors that allow the surgeon to view the retina on a display screen using polarized glasses, digital oculars or a head-mounted display. The display screen provides relief from having to view the surgery using oculars and allows others in the operating room to see exactly as the surgeon does. The system also allows for improved images under high magnification, and increased depth of field compared to a conventional optical, analog surgical microscope, which allow for improved visualization of the eye.

Despite these advances, a common eye condition that is often challenging to visualize and treat effectively is the presence of media opacities. Media opacities are generally caused by microscopic collagen fibers within the vitreous and may result from vitreous syneresis. They can worsen vision quality as they may scatter light entering the eye, thus appearing as spots, shadows, cobwebs, or other assorted shapes that appear to move about in the field of vision of a patient.

More severe cases of media opacities may be treated by laser vitreolysis using a yttrium aluminum garnet (YAG) laser. During this procedure, laser pulses may interact with the eye tissue of a patient to remove the media opacity. However, laser vitreolysis with a YAG laser may have limited effectiveness in the removal of media opacities due to the high energy and low targeting precision of the YAG laser. This may result in a large area of affected tissue, and precise control of the focus and dose of the laser treatment to avoid adverse side effects may be challenging.

### SUMMARY

The present disclosure provides a laser treatment system to improve removal of media opacities for vitreoretinal surgery and associated methods. The laser treatment system includes an optical coherence tomography (OCT) imaging system that generates a plurality of profile depth scans and executes instructions on a processor to detect a position, a volume, or a combination thereof, of a media opacity in an eye based on the plurality of profile depth scans. The laser treatment system also includes a 3D eye tracker that executes instructions on the processor to track the position, the volume, or a combination thereof, of the media opacity in the eye based on the plurality of profile depth scans. The laser treatment system also includes a laser system including a treatment laser and that precisely targets a plurality of ultra-short laser pulses generated by the treatment laser at the media opacity in the eye to at least partially remove the media opacity.

The laser treatment system and its methods of use may include the following additional features: i) the plurality of ultra-short laser pulses may be uniformly targeted within a treatment volume; ii) the system may further include a surgical camera that is a digital camera, an HDR camera, a 3D camera, or any combination thereof; iii) the OCT imaging system may be operable to provide time domain OCT, frequency domain OCT, spectral domain OCT, swept source OCT, OCT angiography, or any combination thereof; iv) the treatment laser may generate a pulse with a time duration of between about a femtosecond (10⁻¹⁵ s) and about 50 picoseconds (50×10⁻¹² s); v) the treatment laser may emit light with a wavelength of about 1,030 nm or about 1,050 nm; vi) the laser system may be a LenSx® Laser (LenSx Lasers, Inc. Corp, California); vii) the laser system may further include a shaping system that modulates a phase of a laser beam to provide a phase-modulated laser beam, a sweeping optical scanner that orients the phase-modulated laser beam to provide a modulated and displaced laser beam and an optical focusing system that displaces a focusing plane of the modulated and displaced laser beam to provide a plurality of cutout planes; viii) the plurality of cutout planes may define a treatment volume; ix) the laser treatment system may be a component of an NGENUITY® 3D Visualization System; x) the 3D eye tracker may provide at least one real time feedback indication about the position and the volume of the media opacity, and provide at least one real time feedback indication to signal if the media opacity has at least been partially removed.

The present disclosure further provides a method for treating a media opacity by identifying a position and a volume of the media opacity using an OCT imaging system; tracking the position and the volume of the media opacity using a 3D eye tracker; using signals about the position and the volume of the media opacity to determine a treatment volume; treating the treatment volume with precise targeting of a plurality of ultra-short laser pulses generated by a treatment laser; and at least partially removing the media opacity.

The present disclosure further provides a method for treating a media opacity by identifying the media opacity using an OCT imaging system; tracking the media opacity using a 3D eye tracker; treating the media opacity according to a treatment plan using a laser system; using at least one real time feedback indication to provide a real time status of the media opacity; and updating the treatment plan in real time in response to the at least one real time feedback indication. The at least one real time feedback indication may include signals about a position and a volume of the media opacity. Updating the treatment plan may include changing a power setting on a treatment laser, changing a duration of a treatment, changing a treatment volume, changing a treatment volume extension, or any combination thereof. Updating the treatment plan may include stopping a treatment.

The present disclosure further provides a method for treating a media opacity by identifying a media opacity using an OCT imaging system; tracking the media opacity using a 3D eye tracker; using at least one calculated feedback indication to determine a treatment plan; and treating the media opacity according to the treatment plan using a laser system. The at least one calculated feedback indication may be based on a prediction by a machine learning algorithm.

The present disclosure further provides a medical system that includes a processor, an OCT imaging system coupled to the processor, a 3D eye tracker coupled to the processor, a laser system, and a memory medium that is coupled to the processor. The memory medium includes instructions that, when executed by the processor, cause the medical system to utilize the OCT imaging system to identify a media opacity in an eye of a patient. The memory medium further includes instructions that, when executed by the processor, cause the medical system to utilize the 3D eye tracker to track a position and a volume of the media opacity. The memory medium further includes instructions that, when executed by the processor, cause the medical system to utilize the laser system to at least partially remove the media opacity in the eye of the patient. The laser system may include a laser that is an ultra-short pulse laser.

Aspects of the laser treatment system and its methods of use may be combined with one another unless clearly mutually exclusive. In addition, the additional features of the laser treatment system and its associated methods described above may also be combined with one another unless clearly mutually exclusive.

### BRIEF DESCRIPTION OF THE DRAWINGS

For a more complete understanding of the present disclosure and its features and advantages, reference is now made to the following description, taken in conjunction with the accompanying drawings, which are not to scale, in which like numerals refer to like features, and in which:

FIG. 1 is a schematic representation of a laser treatment system, including a laser system, a surgical camera, an optical coherence tomography (OCT) imaging system, a 3D eye tracker, a surgical camera system, and a display;

FIG. 2 is a schematic representation of a laser system, including a treatment laser, a laser beam, and a laser control device;

FIG. 3 is a schematic representation of a laser system, including a treatment laser, a laser beam, a laser control device, an optical focusing system, a sweeping optical scanner, and a shaping system;

FIG. 4 is a flow diagram of a method for at least partially removing a media opacity from an eye of a patient;

FIG. 5 is a flow diagram of a method for treating a media opacity using at least one real time feedback indication;

FIG. 6 is a flow diagram of a method for treating a media opacity using at least one calculated feedback indication;

FIG. 7 is a schematic representation of a computer system, including a laser treatment system;

FIGS. 8A-8C are schematic representations of a medical system, including a laser treatment system; and

FIG. 9 is an illustration of a medical system, including a laser treatment system, a surgeon, and a patient.

### DETAILED DESCRIPTION

The present disclosure provides systems including a laser treatment system to improve removal of media opacities and associated methods.

Vitreoretinal surgeons face unique challenges when operating on the internal portions of the eye. For example, a combination of imaging techniques may be necessary to visualize and treat a particular eye disease. One condition that can worsen the visual quality of a patient and is currently challenging to treat is the presence of media opacities. Media opacities, which may also be known as vitreous floaters, are generally caused by microscopic collagen fibers within the vitreous. These may clump and cast shadows on the retina and appear as floaters to the patient. Media opacities may be caused by a shrinkage of the vitreous, a process known as vitreous syneresis or vitreous liquefaction. In a healthy eye, hyaluronic acid may prevent collagen fibrils agglomerating in the vitreous cavity and maintain the transparency of the vitreous. However, as the eye ages, hyaluronic acid may dissociate from collagen. This may cause cross-linking and aggregation of collagen to form fibrous structures that scatter light, and ultimately become media opacities.

Treatment of media opacities may be limited to either a vitrectomy or laser vitreolysis. Vitrectomy may be used in severe cases, and is an invasive procedure that can cause complications, for example, retinal detachment, anterior vitreous detachment and macular edema. Laser vitreolysis is generally performed using an ophthalmic laser that is a yttrium aluminum garnet (YAG) laser. However, the benefit: risk ratio of this treatment is unclear. A YAG laser is generally designed for use in the anterior portion of the eye and may provide a limited view of the vitreous, making identifying a media opacity difficult. Use of a YAG laser may also carry a high risk of damaging surrounding ocular tissue. In particular, the high energy and low targeting precision of the YAG laser may prevent precise control of the focus and dose, resulting in a large area of affected tissue during laser treatment. This may risk damaging the photoreceptor cells of the retina, for example the macular and fovea. The retina is very thin (generally about 200-300 microns in thickness) and it is important to avoid damage to the retina during vitreoretinal surgery.

The systems and methods of the present disclosure may provide for safer and more effective removal of a media opacity for vitreoretinal surgery. The laser treatment systems and methods of the present disclosure may improve removal of a media opacity as compared to current systems and methods by using swept source optical coherence tomography (OCT) to identify a vitreous floater. The laser treatment systems and methods as described herein may improve removal of a media opacity as compared to current systems and methods by tracking the media opacity during treatment using digital eye tracking. The laser treatment systems and methods as described herein may improve removal of a media opacity as compared to current systems and methods by using precise targeting of an ultra-short pulse laser to at least partially remove the media opacity. Precise targeting may reduce the risk of hitting delicate areas such as the retina with the laser. In addition, use of an ultra-short pulse laser may result in minimal or no damage to the back of the eye if the target media opacity is missed. The laser treatment systems and methods as described herein may improve removal of a media opacity as compared to current systems and methods by monitoring the progress of the laser treatment in real time. This may allow for an adjustment of the dosage to provide the lowest dosage necessary to at least partially remove the media opacity. The laser treatment systems and methods as described herein may improve removal of a media opacity as compared to current systems and methods by providing a non-invasive treatment that may be an office-based procedure. The laser treatment systems and methods as described herein may improve removal of a media opacity as compared to current systems and methods by providing a treatment plan involve customizing treatment variables and based on at least one feedback indication.

The systems and methods disclosed herein may improve removal of a media opacity by providing a laser treatment system that may identify, track, and treat a media opacity. The laser treatment system may identify the media opacity using an OCT imaging system. Once the media opacity is identified, the laser treatment system may then track the media opacity using a 3D eye tracker. After the media opacity is identified and tracked, the laser treatment system may use a laser system to treat the media opacity. The laser system may use an ultra-short pulse laser to at least partially remove the media opacity. The laser treatment system may be a component of an ocular-free digital visualization system such as an NGENUITY® (Novartis AG Corp., Switzerland) 3D Visualization System. This visualization system may provide extended depth of field (up to five times greater depth of field as compared to an analog microscope), higher magnification (approximately 48% increased magnification as compared to an analog microscope), higher axial and lateral resolution (approximately 50% greater as compared to an analog microscope), and enhanced stereopsis to facilitate 3D visualization capabilities. These visualization improvements may provide improved removal of a media opacity by the laser treatment system.

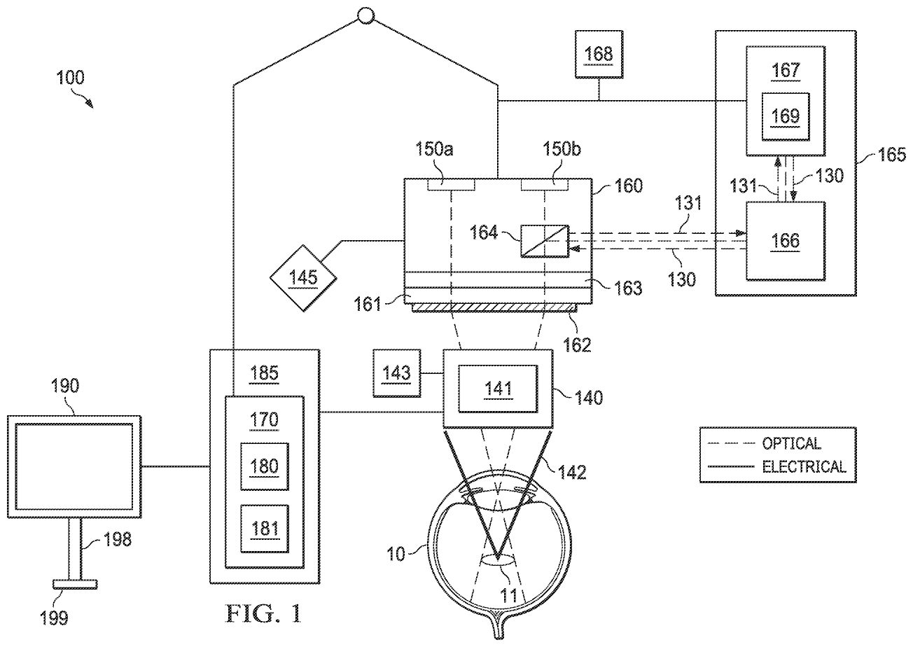

Referring now to FIG. 1, laser treatment system 100 may include laser system 140, surgical camera 160, OCT imaging system 165, 3D eye tracker 168, surgical camera system 185, and display 190.

Laser treatment system 100 as depicted in FIG. 1 is an ocular-free digital visualization system. Laser treatment system 100 may be included as a component of a visualization system such as an NGENUITY® (Novartis AG Corp., Switzerland) 3D Visualization System. Laser treatment system 100 may further include various other electronic and mechanical components in different implementations. Accordingly, while the particular optical design discussed with reference to FIG. 1 is specific to an ophthalmic visualization system that includes surgical camera 160, one skilled in the art will appreciate that alternative optical arrangements to support other ophthalmic visualization systems are within the scope of the disclosure.

Surgical camera 160 may be positioned above eye 10. Surgical camera 160 may be a digital camera, an HDR camera, a 3D camera, or any combination thereof. Surgical camera 160 may include at least one sensor, and may include sensors 150a and 150b. Sensors 150a and 150b may be complementary metal-oxide semiconductor (CMOS) sensors or charge-coupled device (CCD) sensors. Surgical camera 160 may be a monochrome camera, or may be a color camera, and sensors 150a and 150b may be monochrome image sensors or may be color image sensors. Sensors 150a and 150b may capture a digital image using light reflected off eye 10. Sensors 150a and 150b may capture a digital image of eye 10.

Laser treatment system 100 may include visible light illumination source 145, which may provide an illumination source for surgical camera 160. Visible light illumination source 145 may be an endoilluminator (not shown). Visible light illumination source 145 may include a xenon source, a white LED light source, or any other suitable visible light source. Visible light illumination source 145 may illuminate eye 10.

Surgical camera 160 may also utilize optomechanical focus system 161, zoom system 162, and variable working distance system 163. Surgical camera 160 may be communicatively coupled with surgical camera system 185 and display 190. Surgical camera system 185 may include image processing system 170, processor 180, and memory medium 181. Digital images captured by sensors 150a and 150b may be processed by image processing system 170. Image processing system 170 may include processor 180. Sensors 150a and 150b may detect light reflected off eye 10 and send a signal corresponding to the detected light to processor 180. Processor 180 may execute instructions to produce a digital image of eye 10. The digital image of eye 10 may be displayed on display 190. Sensors 150a and 150b may detect light to provide a 3D image of eye 10.

Processor 180 may include, for example, a field-programmable gate array (FPGA), a microprocessor, a microcontroller, a digital signal processor (DSP), a graphics processing unit (GPU), an application specific integrated circuit (ASIC), or any other digital or analog circuitry configured to interpret and/or execute program instructions and/or process data.

Processor 180 may include any physical device able to store and/or execute instructions. Processor 180 may execute processor instructions to implement at least a portion of one or more systems, one or more flow charts, one or more processes, and/or one or more methods described herein. For example, processor 180 may execute instructions to produce a digital image of eye 10. Processor 180 may be configured to receive instructions from memory medium 181. In one example, processor 180 may include memory medium 181. In another example, memory medium 181 may be external to processor 180. Memory medium 181 may store the instructions. The instructions stored by memory medium 181 may be executable by processor 180 and may be configured, coded, and/or encoded with instructions in accordance with at least a portion of one or more systems, one or more flowcharts, one or more methods, and/or one or more processes described herein.

A FPGA may be may be configured, coded, and/or encoded to implement at least a portion of one or more systems, one or more flow charts, one or more processes, and/or one or more methods described herein. For example, the FPGA may be configured, coded, and/or encoded to produce a digital image of eye 10. An ASIC may be may be configured to implement at least a portion of one or more systems, one or more flow charts, one or more processes, and/or one or more methods described herein. For example, the ASIC may be configured, coded, and/or encoded to produce a digital image of eye 10. A DSP may be may be configured, coded, and/or encoded to implement at least a portion of one or more systems, one or more flow charts, one or more processes, and/or one or more methods described herein. For example, the DSP may be configured, coded, and/or encoded to produce a digital image of eye 10.

A single device may include processor 180 and image processing system 170, or processor 180 may be separate from image processing system 170. In one example, a single computer system may include processor 180 and image processing system 170. In another example, a device may include integrated circuits that may include processor 180 and image processing system 170. Alternatively, processor 180 and image processing system 170 may be incorporated into a surgical console.

Processor 180 may interpret and/or execute program instructions and/or process data stored in memory medium 181. Memory medium 181 may be configured in part or whole as application memory, system memory, or both. Memory medium 181 may include any system, device, or apparatus configured to hold and/or house one or more memory devices. Each memory device may include any system, any module or any apparatus configured to retain program instructions and/or data for a period of time (e.g., computer-readable media). One or more servers, electronic devices, or other machines described may include one or more similar such processors or memories that may store and execute program instructions for carrying out the functionality of the associated machine.

Display 190 may be a head-up display mounted on support member 198 and mount base 199. Support member 198 and mount base 199 may be adjustable to change the distance between display 190 and the surgeon. Display 190 may also be ceiling mounted. Display 190 may be communicatively coupled with surgical camera system 185. Display 190 may be a picture-in-picture display. In another example, surgical camera 160 may be a 3D HDR camera and display 190 may be a 3D 4K OLED surgical display. Display 190 may display a digital image of eye 10. Display 190 may display a 3D surgical image of eye 10. Processor 180 may be an ultra-high-speed 3D image processor, which may optimize 3D HDR images in real time.

Surgical camera 160 may be communicatively coupled with surgical camera system 185 and display 190. Display 190 may receive information from surgical camera 160 via surgical camera system 185. Display 190 may display a digital image of eye 10 captured by surgical camera 160.

Eye 10 may include media opacity 11. Media opacity 11 may be located in the vitreous of eye 10. Media opacity 11 may be visualized using OCT imaging system 165. OCT may use near-infrared light to provide high resolution, depth resolved imaging of ocular structures. OCT imaging system 165 may include OCT scanner 166 and OCT controller 167. OCT controller 167 may include a light source, an analyzing unit, or a combination thereof. OCT controller 167 may generate OCT imaging beam 130. OCT scanner 166 may be optically coupled to surgical camera 160. OCT scanner 166 may provide a profile depth scan of the eye tissue of eye 10. The profile depth scan may provide information about the eye tissue of eye 10 that is not readily visible from digital images generated using surgical camera 160. The profile depth scan may provide information about the position of media opacity 11. The profile depth scan may provide information about the volume of media opacity 11. OCT imaging system 165 may generate an OCT image from the profile depth scan. The OCT image may be displayed on display 190. Alternatively, the OCT image may be displayed on another display

OCT imaging system 165 may represent any configuration of OCT instruments and configurations necessary to visualize eye 10. OCT imaging system 165 may represent any configuration of OCT instruments and configurations necessary to visualize media opacity 11. OCT imaging system 165 may provide time domain OCT. OCT imaging system 165 may provide frequency domain OCT, such as, but not limited to, spectral domain OCT, swept source OCT, and OCT angiography. OCT scanner controller 167 may include OCT laser 169. OCT imaging system 165 may be a swept source OCT imaging system. In this example, OCT laser 169 may be a short cavity swept laser, which may sweep across a narrow band of wavelengths with each scan. OCT laser 169 may generate an OCT imaging beam 130 that includes an infrared or near infrared light beam covering a relatively narrow band of wavelengths (for example, 830 nm-870 nm, 790 nm-900 nm, 950 nm-1150 nm, 1000-1300 nm, or 1200-1400 nm). OCT laser 169 may have a wavelength centered at about 1000 nm. Alternatively, OCT laser 169 may have a wavelength centered at about 1300 nm. However, OCT laser 169 may generate an OCT imaging beam 130 having any suitable spectral range of wavelengths. OCT imaging system 165 may be a swept source OCT angiography imaging system. Alternatively, OCT imaging system 165 may be a swept source OCT imaging system such as ANTERION® (Heidelberg Engineering GmbH, Germany).

OCT imaging system 165 may provide at least one profile depth scan of eye 10. Laser treatment system 100 may include a right light beam that is split at partial mirror 164. OCT scanner 166 may control the output of OCT imaging beam 130. Partial mirror 164 may receive OCT imaging beam 130 from OCT scanner 166. A portion of OCT imaging beam 130 reaching eye 10 may be reflected by eye 10 as OCT measurement beam 131. OCT measurement beam 131 may return to OCT imaging system 165 along substantially the same optical path as traveled by OCT imaging beam 130. Partial mirror 164 may output OCT measurement beam 131 to OCT scanner 166. Once OCT measurement beam 131 reaches OCT scanner controller 167, OCT scanner controller 167 may determine a profile depth scan of eye 10. OCT scanner controller 167 may also construct an OCT image based on interference between OCT measurement beam 131 and a reference arm of OCT imaging beam 130, as is known in the art.

OCT scanning controller 167 may output a display image of the OCT image to display 190 using image processing system 170. OCT scanning controller 167 may perform image processing in real time with relatively high refresh rates. This may provide a surgeon with almost instantaneous feedback for viewing and controlling OCT images generated by OCT scanner 166.

As used herein, “real time” may refer to the updating of information at the same rate as data is received. In the context of the laser treatment systems and methods of the present disclosure, “real time” may mean that OCT data is acquired, processed, and transmitted from a photosensor at a high enough data rate and a low enough delay that when the data is displayed, objects more smoothly without user-noticeable judder or latency. For example, this may occur when new OCT images are acquired, processed, and transmitted at a rate of at least 30 frames per second and displayed at about 60 frames per second, and where the combined processing of the signal has less than about a 1/30^{th} second of delay.

Image processing system 170 may execute image processing algorithms on processor 180, for example classification, feature extraction, pattern recognitions, or any combination thereof, on a profile depth scan generated by OCT scanner 166. A profile depth scan generated by OCT scanner 166 may include two-dimensional (2D) scan data and 3D scan data. OCT imaging system 165 may execute instructions on processor 180 to detect the position of media opacity 11 in eye 10 based on at least one profile depth scan. OCT imaging system 165 may execute instructions on processor 180 to detect the volume of media opacity 11 in eye 10 based on at least one profile depth scan. OCT imaging system 165 may alternatively generate a plurality of profile depth scans. OCT imaging system 165 may execute instructions on processor 180 to detect the position, the volume, or a combination thereof, of media opacity 11 in eye 10 based on the plurality of profile depth scans.

OCT scanner 166 may direct OCT imaging beam 130 to a position in eye 10 as directed by the surgeon. OCT scanner 166 may direct OCT imaging beam 130 to a position in eye 10 suitable for identifying media opacity 11. OCT scanner 166 may comprise any suitable optical component or combination of optical components facilitating focusing of OCT imaging beam 130 in the X-Y plane. For example, OCT scanner 166 may include one or more of a pair of scanning mirrors, a micro-mirror device, a microelectromechanical systems (MEMS) device, a deformable platform, a galvanometer-based scanner, a polygon scanner, a resonant PZT scanner, or any combination thereof.

3D eye tracker 168 may track media opacity 11. 3D eye tracker 168 may track media opacity 11 after OCT imaging system 165 has identified media opacity 11. 3D eye tracker 168 may track media opacity 11 using digital eye tracking. 3D eye tracker 168 may track media opacity 11 using pixel-based eye tracking or scanning laser-based eye tracking. 3D eye tracker 168 may track the position, the volume, or a combination thereof of media opacity 11. 3D eye tracker 168 may include any suitable combination of hardware, firmware, and software necessary to track media opacity 11. Alternatively, 3D eye tracker 168 may utilize processor 180 and memory medium 181 of image processing system 170. Eye tracker 168 may execute instructions on processor 180 to track the position, the volume, or a combination thereof, of the media opacity based on the plurality of profile depth scans generated by OCT imaging system 165. For example, processor 180 may receive and process profile depth scans generated by OCT imaging system 165. Memory medium 181 may store the pre-processed profile depth scans, the post-processed profile depth scans, or a combination thereof. Processor 180 may detect the position, the volume, or a combination thereof, of media opacity 11 based on the profile depth scans. Processor 180 may also detect a change in the position, a change in the volume, or a combination thereof, of media opacity 11 based on the profile depth scans. Eye tracker 168 may track media opacity 11 until media opacity 11 has been at least in part removed during surgery. Alternatively, 3D eye tracker 168 may track media opacity 11 for a duration of time specified by the surgeon.

3D eye tracker 168 may provide for direction of OCT imaging beam 130 to a position in eye 10 in an automated manner. For example, OCT scanner 166 may direct OCT imaging beam 130 to a position in eye 10 based on the signals generated 3D eye tracker 168. In another example, OCT scanner 166 may include a pair of scanning mirrors each coupled to a motor drive. The motor drives may to rotate the mirrors about perpendicular axes. By controlling the position of the coupled motors, for example, using signals generated 3D eye tracker 168, the X-Y positioning of OCT imaging beam 130 in eye 10 may be controlled. Alternatively, 3D eye tracker 168 may be disabled in laser treatment system 100. In this case, the position, volume, or a combination thereof, of media opacity 11 may be monitored manually by the surgeon using OCT imaging system 165.

Laser system 140 may treat media opacity 11. Laser system 140 may treat media opacity 11 after OCT imaging system 165 has identified media opacity 11 and 3D eye tracker 168 has tracked media opacity 11. Laser system 140 may include treatment laser 141 and laser control device 143. Treatment laser 141 may provide laser beam 142. Treatment laser 141 may be an ultra-short pulse laser. In one example, treatment laser 141 may be OCT laser 169. Treatment laser 141 may be any laser capable of providing a laser beam 142 that may at least partially remove media opacity 11 during treatment while resulting in minimal or no damage to the back of the eye 10. Laser system 140 may at least partially remove media opacity 11 using photodisruptive laser fragmentation by treatment laser 141.

For example, treatment laser 141 may provide a pulse with a time duration of about a picosecond (10⁻¹² s) or less. In another example, treatment laser 141 may provide a pulse with a time duration of between about a femtosecond (10⁻¹⁵ s) and about a picosecond (10⁻¹² s). In a further example, treatment laser 141 may provide a pulse with a time duration of between about 0.01 picoseconds and 50 picoseconds. In yet another example, treatment laser 141 may provide a pulse with a time duration of between about a femtosecond (10⁻¹⁵ s) and about 50 picoseconds (50×10⁻¹² s).

Laser system 140 may be a LenSx® Laser (LenSx Lasers, Inc. Corp, California). Alternatively, laser system 140 may include treatment laser 141 that is a femtosecond laser, a shaping system, a sweeping optical scanner, and an optical focusing system, as described in U.S. Patent Publication 2019/0159933, filed Oct. 5, 2018, the disclosure of which is incorporated herein by reference in its entirety. Laser system 140 may be controlled by control device 143. For example, control device 143 may control the intensity of treatment laser 141, the focus of laser beam 142, the position of laser beam 142, or any combination thereof.

Laser system 140 may treat media opacity 11 by precisely targeting a plurality of ultra-short laser pulses of laser beam 142 at media opacity 11 to at least partially remove media opacity 11. Each successive ultra-short later pulse may be precisely targeted at media opacity 11 using signals generated by 3D eye tracker 168. Use of eye tracker 168 in combination with the components of surgical camera system 185 in a visualization system such as an NGENUITY® 3D Visualization System may allow highly precise targeting of laser beam 142. In particular, focused femtosecond laser pulses of laser beam 142 may be precisely targeted at media opacity 11 to at least partially remove media opacity 11.

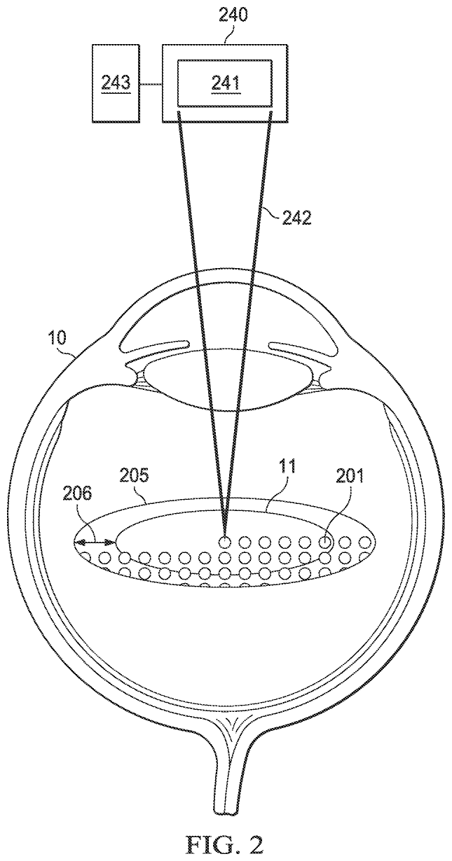

In one example, laser system 240 may treat media opacity 11 as depicted in FIG. 2. Laser system 240 may include treatment laser 241, laser beam 242, and laser control device 243. Treatment laser 241 may provide laser beam 242. Eye tracker 168 may send a signal to laser system 240 to precisely target the focus of laser beam 242 to media opacity 11. Treatment laser 241 may be a femtosecond laser or a picosecond laser and may emit light with a wavelength of about 1,050 nm. In another example, treatment laser 241 may be OCT laser 169. Laser system 240 may treat media opacity 11 by precisely targeting a plurality of ultra-short laser pulses of laser beam 242 at media opacity 11 to at least partially remove media opacity 11. Laser pulses from laser beam 242 may be uniformly targeted within treatment volume 205. Laser system 240 may use signals about the position and the volume of media opacity 11 tracked by 3D eye tracker 168 to determine a treatment volume 205. Treatment volume 205 may be about the same volume as media opacity 11. Alternatively, treatment volume 205 may be larger than media opacity 11 by a volume extension 206. Volume extension 206 may result in a treatment volume that is about 5%, 10%, 15% or 20% larger than the volume of media opacity 11. Alternatively, volume extension 206 may result in any treatment volume necessary to at least partially remove media opacity 11 while resulting in minimal or no damage to the back of the eye 10. Treatment volume 205 may change during treatment if the position and the volume of media opacity 11 changes.

Ultra-short laser pulses from laser beam 242 may result in spots hit by laser 201 within the treatment volume 205. As photodisruptive laser fragmentation generally results in the formation of gas bubbles, laser pulses may be precisely targeted to minimize disruption from the formation of gas bubbles. The laser pulse energy, the position of spots hit by laser 201, and treatment volume 205 may be optimized to achieve the highest effectiveness of breaking up media opacity 11 while minimizing the effects of gas spreading, laser exposure, damage to the back of the eye 10, and procedure time.

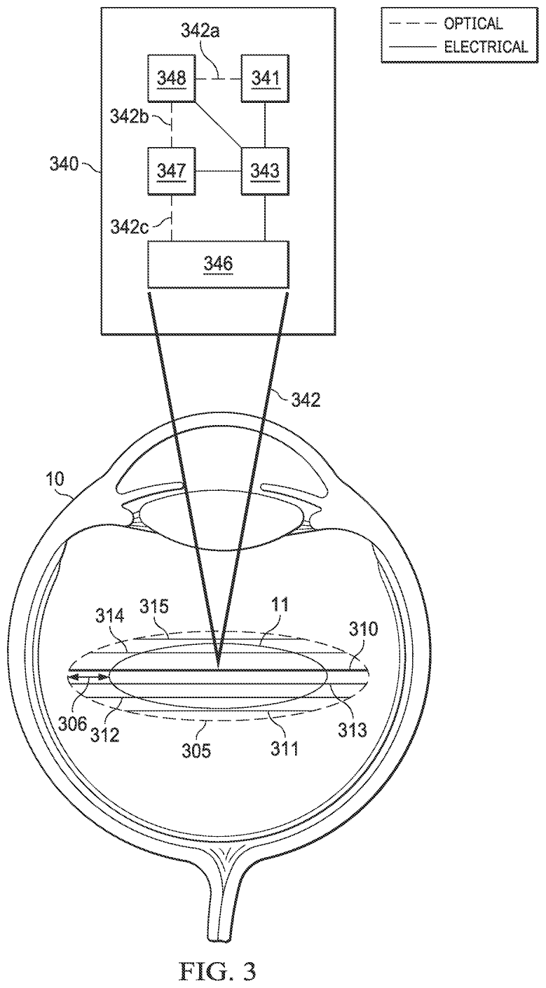

In another example, laser system 340 may treat media opacity 11, as depicted in FIG. 3. Laser system 340 may include treatment laser 341, laser beam 342, laser control device 343, optical focusing system 346, sweeping optical scanner 347, and shaping system 348. Treatment laser 341 may be a femtosecond laser. Treatment laser 341 may emit light with a wavelength of about 1,030 nm in the form of pulses of 400 femtoseconds. Treatment laser 341 may have a power of 20 W and a frequency of 500 kHz. Treatment laser 341 may provide laser beam 342. Laser system 340 may treat media opacity 11 by precisely targeting a plurality of ultra-short laser pulses of laser beam 342 at media opacity 11 to at least partially remove media opacity 11. Laser beam 342 may provide ultra-short laser pulses to a specific treatment volume 305. Treatment volume 305 may be about the same volume as media opacity 11. Alternatively, treatment volume 305 may be larger than media opacity 11 by a volume extension 306. Volume extension 306 may result in a treatment volume that is about 5%, 10%, 15% or 20% larger than the volume of media opacity 11. Alternatively, volume extension 306 may result in any treatment volume necessary to at least partially remove media opacity 11 while resulting in minimal or no damage to the back of the eye 10.

Laser beam 342 may provide ultra-short laser pulses to treatment volume 305 using shaping system 348, sweeping optical scanner 347, and optical focusing system 346. Laser control device 343 may control shaping system 348, sweeping optical scanner 347, and optical focusing system 346. Shaping system 348 may modulate the phase of the laser beam 342a emitted by treatment laser 341. This may distribute the energy of the laser beam to generate plurality of impact points in its focal plane. The plurality of impact points may define a cutout pattern. Shaping system 348 may emit phase-modulated laser beam 342b. Shaping system 348 may include a spatial light modulator. Shaping system 348 may include a liquid crystal on silicon spatial light modulator.

Sweeping optical scanner 347 may orient phase-modulated laser beam 342b emitted by shaping system 348 to displace the cutout pattern along a displacement path that may be in focusing plane 310. The displacement path may be predefined by the surgeon. Sweeping optical scanner 347 may emit modulated and displaced laser beam 342c. Sweeping optical scanner 347 may include at least one optical minor. The at least one optical mirror may pivot about at least two axes in order to orient phase-modulated laser beam 342b.

Optical focusing system 346 may displace focusing plane 310 of modulated and displaced laser beam 342c. This may provide cutout plane 311, 312, 313, 314, or 315. Optical focusing system 346 may include at least one motor-driven lens that may be displaced in the optical path of laser beam 342c.

Thus, shaping system 348 may allow the simultaneous generation of several impact points that define a cutout pattern, sweeping optical scanner 347 may allow the displacement of the cutout pattern in focusing plane 310, and optical focusing system 346 may allow the displacement of the focusing plane 310 in depth to generate cutouts in successive planes 311, 312, 313, 314 and 315. Cutout planes 311, 312, 313, 314 and 315 may define treatment volume 305.

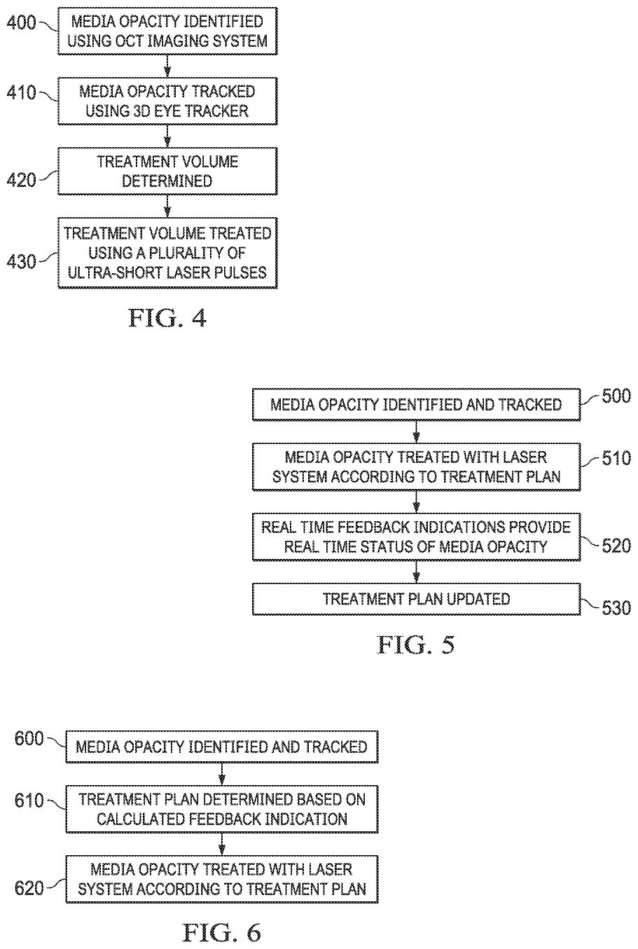

FIG. 4 presents a flow diagram of a method of at least partially removing a media opacity from an eye of a patient. In step 400, a media opacity, such as media opacity 11, is identified using an OCT imaging system, such as OCT imaging system 165. The OCT imaging system may identify a position and a volume of the media opacity. In step 410, the media opacity identified using the OCT imaging system is tracked using a 3D eye tracker, such as 3D eye tracker 168. This step may involve the 3D eye tracker executing instructions on a processor, such as processor 180, to track the position, the volume, or a combination thereof, of the media opacity based on a plurality of profile depth scans generated by the OCT imaging system. In step 420, a treatment volume, such as treatment volume 205, is determined. The treatment volume may be determined using signals about the position and volume of the media opacity detected by the OCT imaging system and tracked by 3D eye tracker. In step 430, the treatment volume may be treated by precise targeting of a plurality of ultra-short laser pulses to at least partially remove the media opacity, which may include precise targeting of focused femtosecond laser pulses. This method may allow the surgeon to at least partially remove the media opacity while resulting in minimal or no damage to the back of the eye of the patient. In addition, as the treatment is non-invasive, it may be an office-based procedure.

During laser surgery using laser treatment system 100, an OCT imaging system such as OCT imaging system 165, a 3D eye tracker such as 3D eye tracker 168, or a combination thereof, may provide at least one real time feedback indication of the progress of the treatment. A real time feedback indication may include an OCT image to provide the status of media opacity 11. An OCT image may be provided during treatment by a laser beam, such as laser beam 142, laser beam 242, or laser beam 342, after treatment by a laser beam has concluded, or a combination thereof. A real time feedback indication may also include signals about the position and volume of media opacity 11 identified by OCT imaging system 165 and tracked by 3D eye tracker 168. A real time feedback indication may be provided to the surgeon to signal that media opacity 11 has at least partially been removed.

Before or during laser surgery using laser treatment system 100, at least one calculated feedback indication may be provided. A calculated feedback indication may include, for example, a suggested treatment volume based on previous patient outcomes for particular media opacity dimensions and volumes, a suggested duration of the treatment based on previous patient outcomes for a particular laser power setting of the treatment laser, a suggested laser power setting of the treatment laser based on a prediction from a machine learning algorithm developed from a test set of pre-operative OCT images and post-operative OCT images from previous laser surgeries, or any combination thereof.

Media opacity 11 may be treated using laser treatment system 100 according to a treatment plan. A treatment plan may involve customizing treatment variables including, but not limited to, the power settings of the treatment laser, the duration of the treatment, the treatment volume, the volume extension, or any combination thereof. Treatment variables may be determined by considering treatment factors including, but not limited to, the volume of the eye of the patient, the volume of the media opacity, the dimensions of the media opacity, the age of the patient, the medical history of the patient, any feedback indications, pre-operative OCT images of the eye of the patient, post-operative OCT images of the eye of the patient, patient surveys, recurrence rate of media opacity after treatment, effectiveness of previous treatments, or any combination thereof.

A treatment plan may be based, at least in part, on at least one real time feedback indication, for example, an OCT image provided during treatment or signals about the position and volume of media opacity 11. The at least one real time feedback indication may be used by the surgeon to increase or decrease the power settings of the treatment laser, for example treatment laser 141, during the laser treatment. The at least one real-time feedback indication may also allow the surgeon to update the treatment plan or stop the treatment in real time when media opacity 11 has at least partially been removed. This may allow the surgeon to use a minimum amount of laser power to at least partially remove a media opacity. In addition, this provide a desired treatment result with minimal risk of damage to the eye of the patient.

FIG. 5 presents a flow diagram for a method for treating a media opacity using at least one real time feedback indication according to the disclosure. In step 500, a media opacity is identified and tracked. The media opacity may be identified by an OCT imaging system such as OCT imaging system 165. The media opacity may be tracked by an eye tracker such as 3D eye tracker 168. In step 510, the media opacity is treated with a laser system, such as laser system 140, laser system 240, or laser system 340, according to a treatment plan. In step 520, during laser treatment, at least one real time feedback indication may provide the real time status of the media opacity. A real time feedback indication may include signals about the position and volume of a media opacity detected by OCT imaging system 165 and tracked by 3D eye tracker 168. In step 530, the surgeon may update the treatment plan in real time in response to the at least one real time feedback indication. The treatment plan may be updated by changing the power settings of the treatment laser, the duration of the treatment, the treatment volume, the volume extension, or any combination thereof. In another example, the surgeon may stop the treatment if the at least one real time feedback indication suggests that the volume of the media opacity is about zero, indicating that the media opacity has been at least partially removed. This may allow the surgeon to use a minimum amount of laser power to at least partially remove the media opacity.

In a further example, a treatment plan may be determined, at least in part, based on at least one calculated feedback indication. The at least one calculated feedback indication may include a suggested value for a treatment variable based on a prediction by a machine learning algorithm developed from a test set of data. The test set of data may include at least one treatment factor. For example, a suggested value for a treatment variable may be based on a prediction by a machine learning algorithm developed from a test set of pre-operative OCT images and post-operative OCT images. In another example, a machine learning algorithm may provide predictions about the optimal power settings of the treatment laser to remove a media opacity of a particular volume. A treatment plan may also be based, at least in part, on a combination of real-time feedback indications and calculated feedback indications.

FIG. 6 presents a flow chart for a method for treating a media opacity using at least one calculated feedback indication according to the disclosure. In step 600, a media opacity is identified and tracked. The media opacity may be identified by an OCT imaging system such as OCT imaging system 165. The media opacity may be tracked by an eye tracker such as 3D eye tracker 168. In step 610, a treatment plan is determined based on at least one calculated feedback indication. An example of a calculated feedback indication may include a suggested value for a treatment variable based on a prediction by a machine learning algorithm developed from a test set of data. In step 620, the media opacity is treated with a laser system, such as laser system 140, laser system 240, or laser system 340, according to the treatment plan determined based on the at least one calculated feedback indication.

Laser treatment system 100 may be used in combination with a computer system 700, as depicted in FIG. 7. Computer system 700 may include a processor 710, a volatile memory medium 720, a non-volatile memory medium 730, and an input/output (I/O) device 740. Volatile memory medium 720, non-volatile memory medium 730, and I/O device 740 may be communicatively coupled to processor 710.

The term “memory medium” may mean a “memory”, a “storage device”, a “memory device”, a “computer-readable medium”, and/or a “tangible computer readable storage medium”. For example, a memory medium may include, without limitation, storage media such as a direct access storage device, including a hard disk drive, a sequential access storage device, such as a tape disk drive, compact disk (CD), random access memory (RAM), read-only memory (ROM), CD-ROM, digital versatile disc (DVD), electrically erasable programmable read-only memory (EEPROM), flash memory, non-transitory media, or any combination thereof. As shown in FIG. 7, non-volatile memory medium 730 may include processor instructions 732. Processor instructions 732 may be executed by processor 710. In one example, one or more portions of processor instructions 732 may be executed via non-volatile memory medium 730. In another example, one or more portions of processor instructions 732 may be executed via volatile memory medium 720. One or more portions of processor instructions 732 may be transferred to volatile memory medium 720.

Processor 710 may execute processor instructions 732 in implementing at least a portion of one or more systems, one or more flow charts, one or more processes, and/or one or more methods described herein. For example, processor instructions 732 may be configured, coded, and/or encoded with instructions in accordance with at least a portion of one or more systems, one or more flowcharts, one or more methods, and/or one or more processes described herein. Although processor 710 is illustrated as a single processor, processor 710 may be or include multiple processors. One or more of a storage medium and a memory medium may be a software product, a program product, and/or an article of manufacture. For example, the software product, the program product, and/or the article of manufacture may be configured, coded, and/or encoded with instructions, executable by a processor, in accordance with at least a portion of one or more systems, one or more flowcharts, one or more methods, and/or one or more processes described herein.

Processor 710 may include any suitable system, device, or apparatus operable to interpret and execute program instructions, process data, or both stored in a memory medium and/or received via a network. Processor 710 further may include one or more microprocessors, microcontrollers, DSPs, ASICs, or other circuitry configured to interpret and execute program instructions, process data, or both.

I/O device 740 may include any instrumentality or instrumentalities, which allow, permit, and/or enable a user to interact with computer system 700 and its associated components by facilitating input from a user and output to a user. Facilitating input from a user may allow the user to manipulate and/or control computer system 700, and facilitating output to a user may allow computer system 700 to indicate effects of the user's manipulation and/or control. For example, I/O device 740 may allow a user to input data, instructions, or both into computer system 700, and otherwise manipulate and/or control computer system 700 and its associated components. I/O devices may include user interface devices, such as a keyboard, a mouse, a touch screen, a joystick, a handheld lens, a tool tracking device, a coordinate input device, or any other I/O device suitable to be used with a system.

I/O device 740 may include one or more buses, one or more serial devices, and/or one or more network interfaces, among others, that may facilitate and/or permit processor 710 to implement at least a portion of one or more systems, processes, and/or methods described herein. In one example, I/O device 740 may include a storage interface that may facilitate and/or permit processor 710 to communicate with an external storage. The storage interface may include one or more of a universal serial bus (USB) interface, a SATA (Serial ATA) interface, a PATA (Parallel ATA) interface, and a small computer system interface (SCSI), among others. In a second example, I/O device 740 may include a network interface that may facilitate and/or permit processor 710 to communicate with a network. I/O device 740 may include one or more of a wireless network interface and a wired network interface. In a third example, I/O device 740 may include one or more of a peripheral component interconnect (PCI) interface, a PCI Express (PCIe) interface, a serial peripheral interconnect (SPI) interface, and an inter-integrated circuit (I2C) interface, among others. In a fourth example, I/O device 740 may include circuitry that may permit processor 710 to communicate data with one or more sensors. In a fifth example, I/O device 740 may facilitate and/or permit processor 710 to communicate data with one or more of a display 750 and laser treatment system 100, among others. As shown in FIG. 7, I/O device 740 may be coupled to a network 770. For example, I/O device 740 may include a network interface.

Network 770 may include a wired network, a wireless network, an optical network, or any combination thereof. Network 770 may include and/or be coupled to various types of communications networks. For example, network 770 may include and/or be coupled to a local area network (LAN), a wide area network (WAN), an Internet, a public switched telephone network (PSTN), a cellular telephone network, a satellite telephone network, or any combination thereof. A WAN may include a private WAN, a corporate WAN, a public WAN, or any combination thereof.

Although FIG. 7 illustrates computer system 700 as external to laser treatment system 100, laser treatment system 100 may include computer system 700. For example, processor 710 may be or include processor 180.

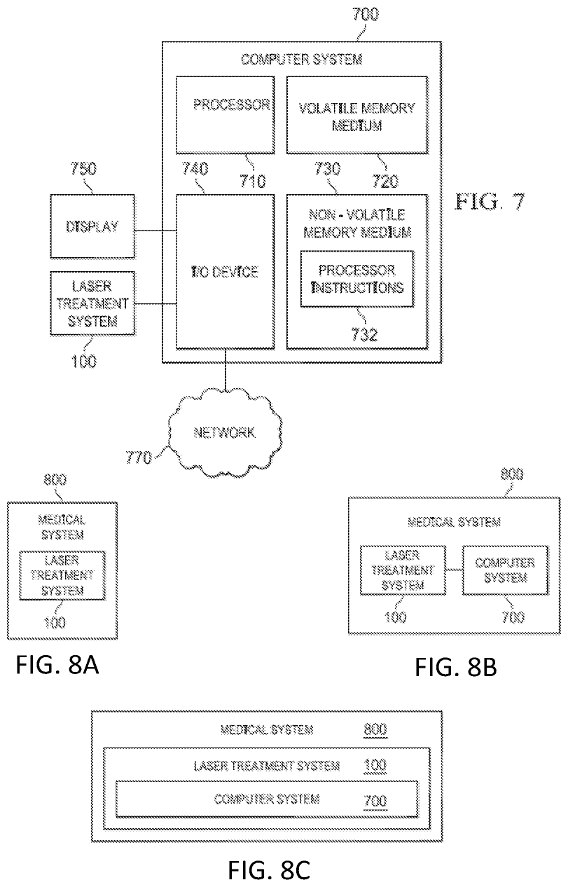

FIGS. 8A-8C illustrate examples of medical system 800. As shown in FIG. 8A, medical system 800 may include laser treatment system 100. As illustrated in FIG. 8B, medical system 800 may include laser treatment system 100 and computer system 700. Laser treatment system 100 may be communicatively coupled with computer system 700. As shown in FIG. 8C, medical system 800 may include laser treatment system 100, which may include computer system 700.

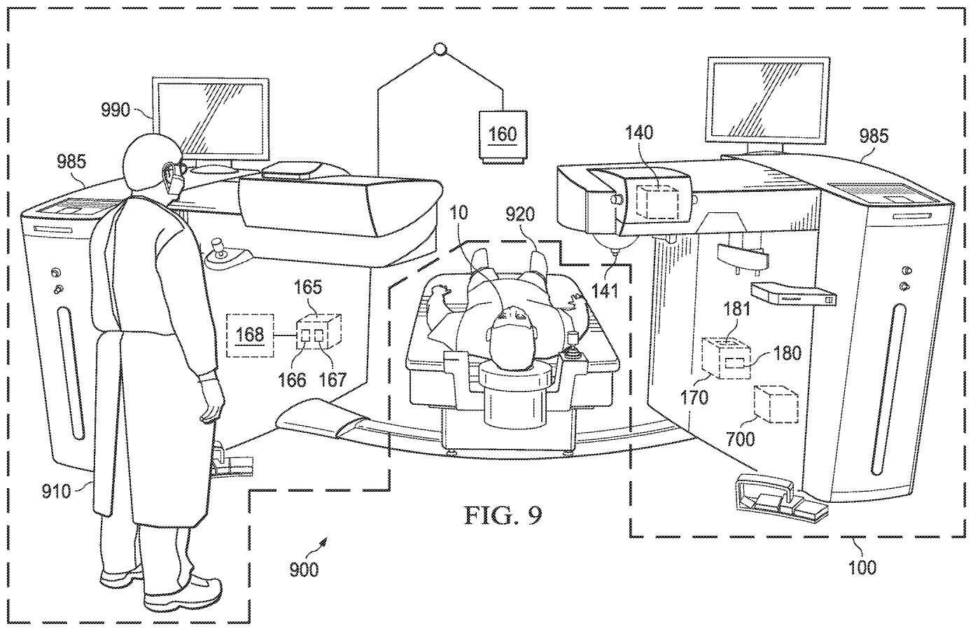

Laser treatment system 100 may be used as a component of medical system 900, as shown in FIG. 9. Medical system 900 may include laser treatment system 100, which may be included in surgical console 985. Medical system 900 may include computer system 700. Laser treatment system 100 may be communicatively coupled with computer system 700. Surgeon 910 may view a digital image of eye 10 of patient 920 on display 990 using surgical camera 160, OCT imaging system 165, or a combination thereof. Surgeon 910 may identify a media opacity in eye 10 using OCT imaging system 165. Surgeon 910 may track a media opacity in eye 10 using 3D eye tracker 168. Surgeon 910 may treat a media opacity in eye 10 using laser system 140.

Including laser treatment system 100 in medical system 900 may allow surgeon 910 to identify, track, and treat a media opacity in eye 10, which may improve removal of a media opacity for vitreoretinal surgery compared to removal without laser treatment system 100. Medical system 900 may include a laser system 140; an OCT imaging system 165, a 3D eye tracker 168, an image processing system 170, a processor 180; and a memory medium 181, such as those in laser treatment system 100. The memory medium 181 may be coupled to the processor 180, and may include instructions that when executed by the processor, cause the medical system to utilize laser treatment system 100, under supervision of surgeon 910, to at least partially remove a media opacity in the eye 10 of patient 920. Although FIG. 9 illustrates computer system 700 as included in laser treatment system 100, computer system 700 may be external to laser treatment system 100. For example, processor 710 may be or include processor 180, or processor 710 may be separate to processor 180.

Laser treatment system 100, computer system 700, medical system 800, medical system 900, and components thereof may be combined with other elements of treatment tools and systems described herein unless clearly mutually exclusive. For instance, laser treatment system 100 may be combined with medical system 800, and may be used with other visualization systems, computer systems and medical systems described herein.

The above disclosed subject matter is to be considered illustrative, and not restrictive, and the appended claims are intended to cover all such modifications, enhancements, and other embodiments which fall within the true spirit and scope of the present disclosure. For example, although a laser treatment system is most commonly needed to improve removal of media opacities for vitreoretinal surgery, if it were useful in another procedure, such as a purely diagnostic procedure not otherwise considered to be surgery, the systems and methods described herein may be employed.

1. A laser treatment system comprising:
an optical coherence tomography (OCT) imaging system operable to:
generate a plurality of profile depth scans;
execute instructions on a processor to detect a position, a volume, or a combination thereof, of a media opacity in an eye based on the plurality of profile depth scans;
a 3D eye tracker operable to:
execute instructions on the processor to track the position, the volume, or a combination thereof, of the media opacity in the eye based on the plurality of profile depth scans; and
a laser system comprising a treatment laser and operable to:
precisely target a plurality of ultra-short laser pulses generated by the treatment laser at the media opacity in the eye to at least partially remove the media opacity.

2. The laser treatment system of claim 1, wherein the plurality of ultra-short laser pulses are uniformly targeted within a treatment volume.

3. The laser treatment system of claim 1, further comprising a surgical camera that is a digital camera, an HDR camera, a 3D camera, or any combination thereof.

4. The laser treatment system of claim 1, wherein the OCT imaging system is operable to provide time domain OCT, frequency domain OCT, spectral domain OCT, swept source OCT, OCT angiography, or any combination thereof.

5. The laser treatment system of claim 1, wherein the treatment laser generates a pulse with a time duration of between about a femtosecond (10⁻¹⁵ s) and about 50 picoseconds (50×10⁻¹² s).

6. The laser treatment system of claim 1, wherein the treatment laser emits light with a wavelength of about 1,030 nm or about 1,050 nm.

7. The laser treatment system of claim 1, wherein the laser system is a LenSx® Laser.

8. The laser treatment system of claim 1, wherein the laser system further comprises:
a shaping system operable to modulate a phase of a laser beam to provide a phase-modulated laser beam;
a sweeping optical scanner operable to orient the phase-modulated laser beam to provide a modulated and displaced laser beam; and
an optical focusing system operable to displace a focusing plane of the modulated and displaced laser beam to provide a plurality of cutout planes.

9. The laser treatment system of claim 8, wherein the plurality of cutout planes define a treatment volume.

10. The laser treatment system of claim 1, wherein the laser treatment system is a component of an NGENUITY® 3D Visualization System.

11. The laser treatment system of claim 1, wherein the 3D eye tracker is further operable to:
provide at least one real time feedback indication about the position and the volume of the media opacity; and
provide at least one real time feedback indication to signal if the media opacity has at least been partially removed.

12. A method for treating a media opacity, the method comprising:
identifying a position and a volume of a media opacity using an OCT imaging system;
tracking the position and the volume of the media opacity using a 3D eye tracker;
using signals about the position and the volume of the media opacity to determine a treatment volume;
treating the treatment volume with precise targeting of a plurality of ultra-short laser pulses generated by a treatment laser; and
at least partially removing the media opacity.

13. A method for treating a media opacity, the method comprising:
identifying a media opacity using an OCT imaging system;
tracking the media opacity using a 3D eye tracker;
treating the media opacity according to a treatment plan using a laser system;
using at least one real time feedback indication to provide a real time status of the media opacity; and
updating the treatment plan in real time in response to the at least one real time feedback indication.

14. The method of claim 13, wherein the at least one real time feedback indication comprises signals about a position and a volume of the media opacity.

15. The method of claim 13, wherein updating the treatment plan comprises changing a power setting on a treatment laser, changing a duration of a treatment, changing a treatment volume, changing a treatment volume extension, or any combination thereof.

16. The method of claim 13, wherein updating the treatment plan comprises stopping a treatment.

17. A method for treating a media opacity, the method comprising:
identifying a media opacity using an OCT imaging system;
tracking the media opacity using a 3D eye tracker;
using at least one calculated feedback indication to determine a treatment plan; and
treating the media opacity according to the treatment plan using a laser system.

18. The method of claim 17, wherein the at least one calculated feedback indication is based on a prediction by a machine learning algorithm.

19. A medical system comprising:
a processor;
an OCT imaging system coupled to the processor;
a 3D eye tracker coupled to the processor;
a laser system; and
a memory medium that is coupled to the processor and that includes instructions, when executed by the processor, cause the medical system to:
utilize the OCT imaging system to identify a media opacity in an eye of a patient;
utilize the 3D eye tracker to track a position and a volume of the media opacity; and
utilize the laser system to at least partially remove the media opacity in the eye of the patient.

20. The medical system of claim 19, wherein the laser system comprises a laser that is an ultra-short pulse laser.
